(19) United States
(12) Patent Application Publication
(10) Pub. No.: US 2013/0335545 A1
(43) Pub. Date: Dec. 19, 2013

(54) SYSTEM FOR INTEGRATED WOUND ANALYSIS

(71) Applicant: Matthew Ross Darling, O'Connor (AU)

(72) Inventor: Matthew Ross Darling, O'Connor (AU)

(21) Appl. No.: 13/995,719

(22) PCT Filed: Dec. 19, 2011
(86) PCT No.: PCT/AU2011/001637

§ 371 Date: Sep. 10, 2013

Related U.S. Application Data
(60) Provisional application No. 61/424,644, filed on Dec. 19, 2010.

(51) Int. Cl. A61B 5/00

(52) U.S. Cl. CPC A61B 5/004
USPC 348/77

(57) ABSTRACT

A system for integrated wound analysis; said system including sensing and image recording elements; sensed data and images of at least a first recording session stored for analysis; said system including a reference system whereby sensing and image recording of any subsequent said recording session substantially repeats sensing and recording of parameters of said first recording session.

### BACKGROUND

The ability to measure temperature, color changes, size and surface contours of a wound exists in the art. Traditionally management and assessment of wounds is done manually by health care professionals.

This involves visual inspection and the taking of notes. Some tools are known in the art to aid in evaluation. These include transparent media, onto which the circumference of wounds are traced. The media are then scanned and compared in series to detect growth or contraction in the wound area. This approach is variable and hard to repeat exactly due to the lack of a visual record. It also interferes with the wound and entails infection risk.

Increasingly, digital cameras are used in the art to keep record of wounds over time. Digital cameras record only a two dimensional image and are subject to variability in ambient light; adversely effecting colour rendition and consistency.

Colour is one of the principle means by which infection is recognised in the art. Specially designed cameras seek to achieve scale consistency in image capture through the use of Doppler radar range finding, however there is no means of assuring consistent viewing angle or detecting swelling within the wound perimeter. In some devices lasers are also used to measure distance from the camera and changes in the surface depth or surface contour of the item being photographed.

Devices to record high-resolution images of changes in surface temperature and surface contours also exist in the art, but this technology has been focused on satellite surveillance and has not been adapted to small scale use.

The disclosed invention is designed to bring the advantages of these technologies and techniques together in one device.

### FIGURES

FIG. 1—Example embodiment positioning.

FIG. 2—Example of viewfinder guided position verification.

FIG. 3—Example of distance measurement and wound surface analysis.

FIG. 4—Example of positioning verification using a marker.

FIG. 5—Example of ambient temperature and light conditions measurement.

FIG. 6—Example of flash based light balance adjustment.

FIG. 7—Example of image and data results being displayed.

FIG. 8—Example of color imaging.

FIG. 9—Example of thermal imaging.

FIG. 10—Example of image set analysis.

FIG. 11—Example of visual analysis display.

FIG. 12—Example of progress analysis display.

FIG. 13—Example of analytical data results being displayed.

### DESCRIPTION AND OPERATION

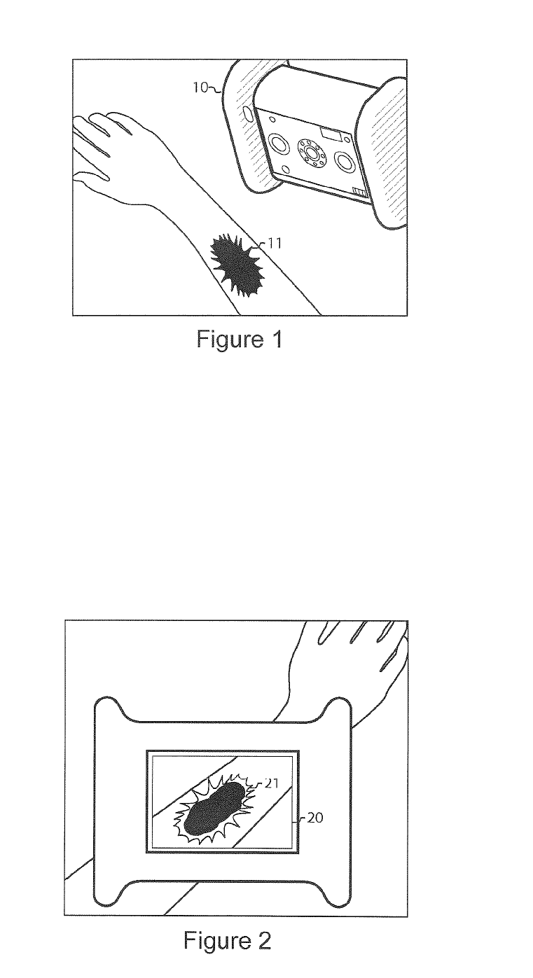

FIG. 1 shows the example embodiment 10 being placed in position to commence analysis over the wound 11. The device has to be placed close enough to the target wound to ensure clear high resolution imagery is available and also to maximize the effectiveness of other wound analysis components in the device. In this example the wound 11 is on a patients right forearm.

FIG. 2 shows a viewfinder screen 20, positioned to give the operator a clear image of the wound. The viewfinder 20 is used to verify that the wound 21 is in frame and can be easily captured and analysed by the device 10.

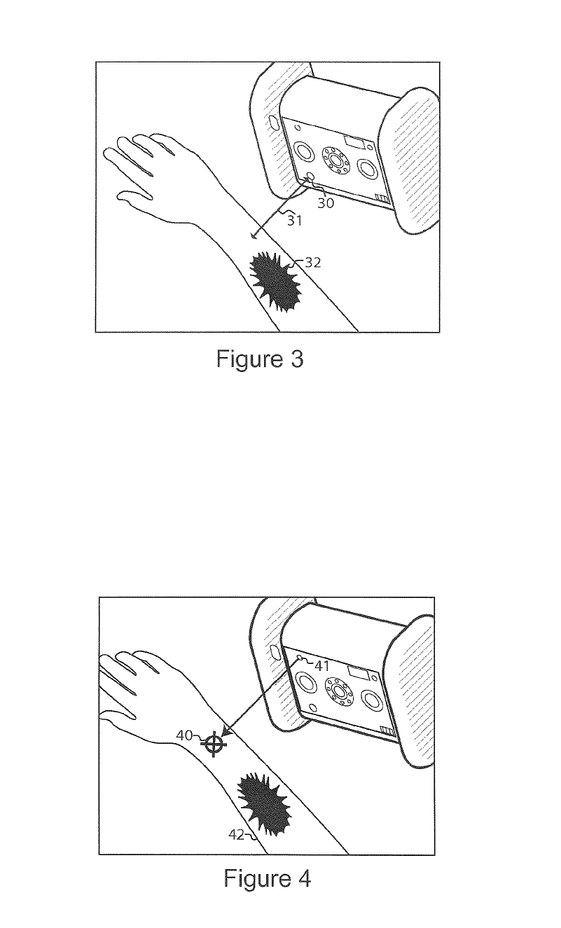

FIG. 3 shows a sensor 30 which is designed to measure the distance 31 from the wound 32 thereby establishing a distance parameter upon which other variables can be calculated such as changes in surface contours. This sensor 30 is also used to provide three dimensional imaging of the wound 32 detecting swelling and providing an assessment of wound's 32 relative size

FIG. 4 shows a reference marker 40 being visually projected by a laser 41 onto the arm 42. This reference marker 40 can be visually seen and measured by the device. The circular design and cross hairs can be used to measure if the source projector 41 is at a different angle or distance from previous analysis sessions.

The size of this reference marker 40 combined with the measurement done as described in FIG. 3, allows for an accurate calculation of variables such as distance and angle, and eliminates erroneous diagnosis due to a difference between measurements taken at various times during the treatment process.

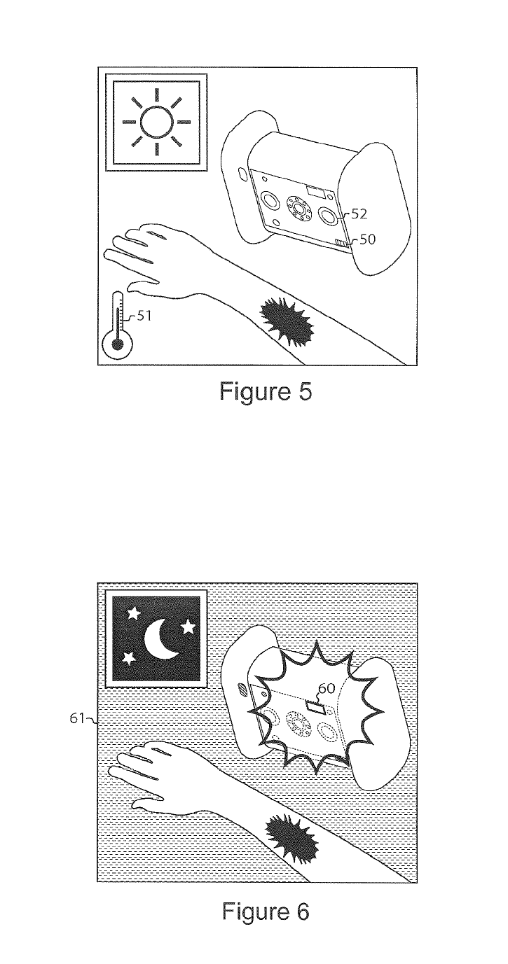

FIG. 5 shows a infra red temperature sensor 50 measuring the ambient temperature of the room 51. This is used to establish a baseline for other measurements which rely on temperature readings related to the wound and surrounding body surface.

At the same time an optical sensor 52 measures the light level and hue, allowing these variables to be taken into account when diagnosing skin discoloration in and around the wound.

FIG. 6 shows a self-adjusting flash 60 which uses the light level measurement taken as described in FIG. 5 and uses this data to ensure an optimal and consistent light balance for color evaluation across all data collected relative to a single wound.

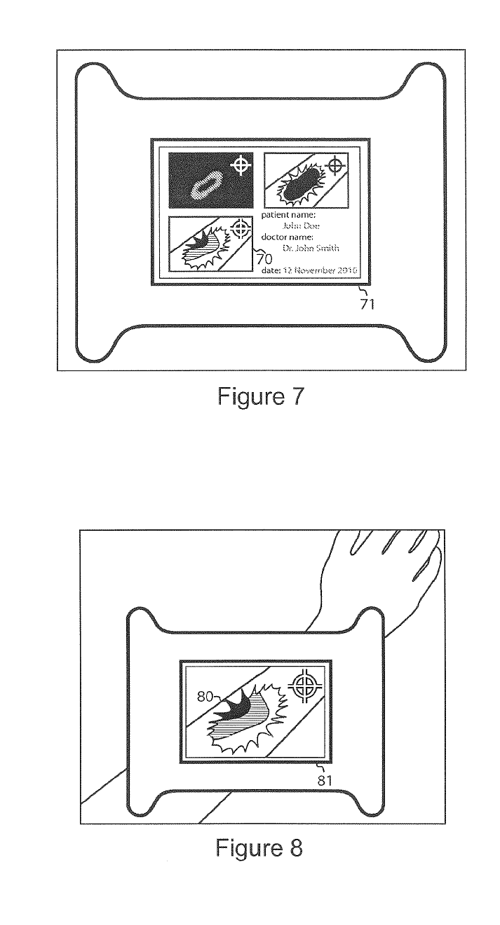

FIG. 7 shows a first set of images 70 being displayed after capture. The device displays the results on the screen 71 and saves the image-set together with a patient identifier, time, date, distance and ambient temperature as measured. This grouped information is used collectively to compare with results from other sessions of grouped data taken at other times and used to analyse what is happening to the wound.

FIG. 8 shows how wound colors 80 are recorded in the set of images and displayed on the screen 81. One example of how wound color is used in wound management is to determine the progress of a bruise where discoloration is clearly a sign of the progress or decay of the wound.

FIG. 9 illustrates a thermal image 90 of the wound being displayed on the screen 91. Wound temperatures are measured by the sensor 50 as described in FIG. 5. The measured temperatures are recorded in an image set. Small variations in temperature on the wound surface are recorded and help in the assessment of many wound conditions including but not limited to signs of tissue death, known in the art as necrosis, and infection.

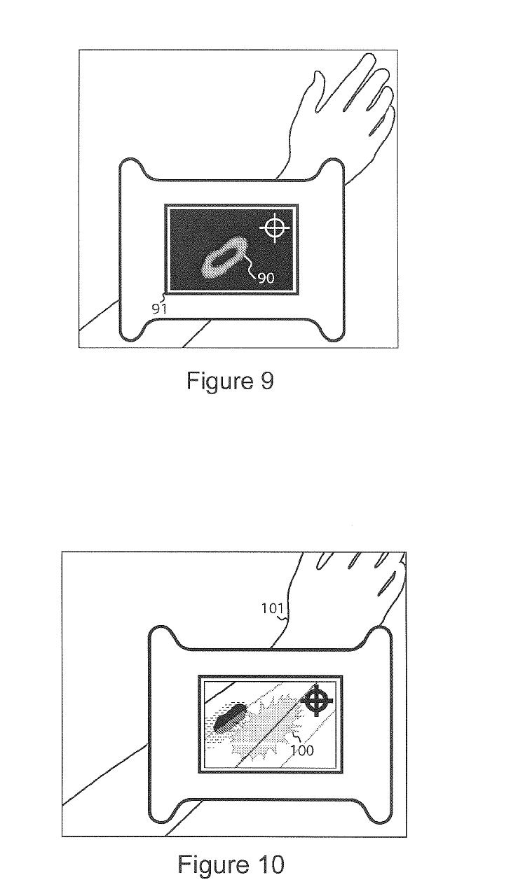

FIG. 10 shows an example of how the set of images 100 can be compiled and presented as a semi transparent layer 100 on top of real-time imagery 101 of the wound and can be analysed by the device.

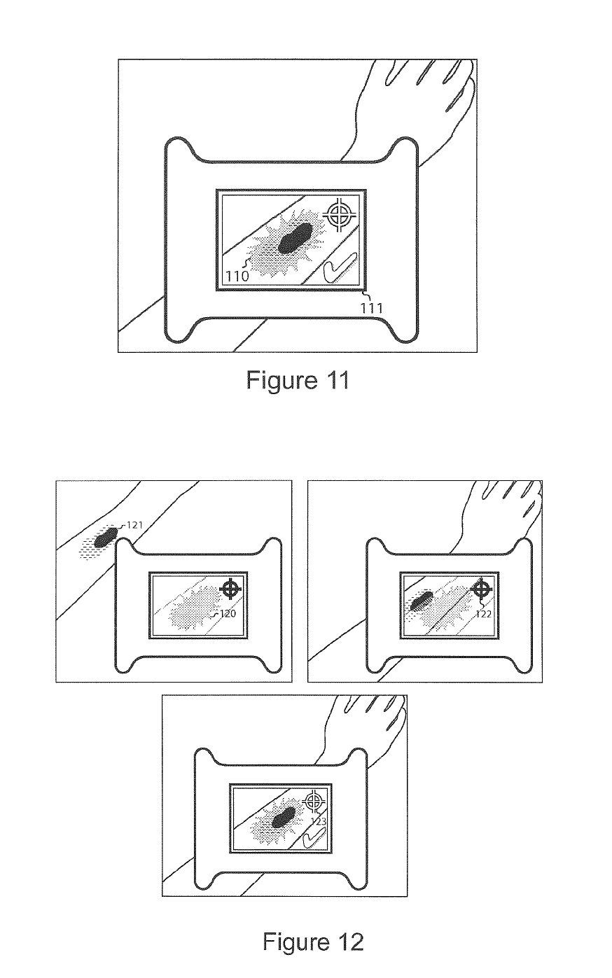

FIG. 11 shows the analysis and compiled images 110 being displayed on the screen 111 as a semi transparent layer which then allows the the operator to make clinical treatment decisions based on the comparison of the previous data and image set with the current condition of the wound.

FIG. 12 shows how, when subsequent images are taken at later dates for diagnosis of the healing progress, the device can be used to monitor this progress. The device retrieves data from the previous patient assessment. The same distance and aspect from the wound are achieved using the saved distance measurement and projected marker as described in FIGS. 1 to 4.

The user is guided by a semi-transparent version of the previous images 120 to adjust the position of the device over the wound 121. When the marker 122 in the saved image 120 is aligned with the marker shown in current diagnosis 123, the steps described in FIG. 5 through to 10 are repeated for a comparative diagnosis.

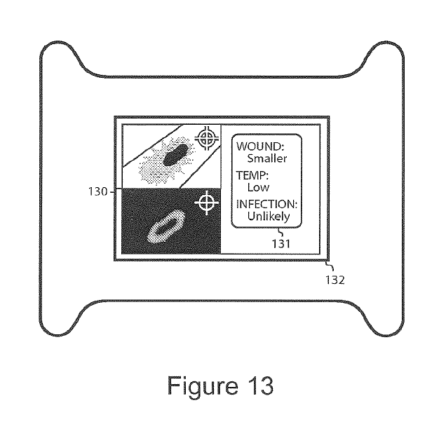

FIG. 13 shows that the device has analysed changes in color, temperature and relative size of the wound 130. Analytical data is then displayed 131 on the screen 132 to assist the operator. In this example, analysis 131 has determined that the wound is smaller and that the surface temperature of the wound has reduced and deduced that the chance of infection is unlikely. All data is saved with patient identification for records, analysis and ongoing treatment.

### Alternate Embodiments

In the example embodiment all the components for analysis are in the one device. An alternative embodiment could have these components separated but connected to one central data processing unit. For example multiple analysis devices of the same type could be used at different times but the results could be coordinated to achieve the same synchronised diagnosis.

In the example embodiment the all measurements required for diagnosis are taken in one session. In an alternative embodiments measurements could be taken continuously or at intervals of any length.

In the example embodiment images are taken at high definition quality commonly used in digital cameras. An alternative embodiment could use much higher resolution, allowing diagnosis even up to microscopic levels.

In the example embodiment the projected reference marker described in FIG. 4 is round with a cross mark. In an alternative embodiment a different size or shape marker than that used in the drawings could be used with the intent of being able to determine changes in size and angle.

The example embodiment uses changes in color, heat, size and contour of the wound to make an analysis. An alternative embodiment could use just three of these to perform an analysis.

The example embodiment is a single, purpose designed module that can be cleaned to minimise infection risk, An alternative embodiment could see the functionality separated out into separate modules. While this may be harder to sanitise it may also deliver advantages in terms of ease of replacement with component failure.

The example embodiment takes temperature measurements and three dimensional images simultaneously, allowing multiple evaluations to be conducted to enable an accurate clinical appraisal. An alternative embodiment could collect measurements from approximately the same time, using multiple devices and still deliver relatively usable analysis.

1-44. (canceled)

45. A wound monitoring device for integrated wound analysis; said device including sensing and image recording elements; sensed data and images of at least a first recording session stored for analysis; said system including a reference system whereby sensing and image recording of any subsequent said recording session substantially repeats sensing and recording of parameters of said first recording session; said sensing and image recording elements including a distance sensor; said distance sensor determining a distance between said device and a reference mark projected by said device onto a surface adjacent said wound.

46. The device of claim 45, wherein said recording parameters of said first recording session include location and disposition of said sensing and image recording elements relative a subject wound.

47. The device of claim 45, wherein said recording parameters further include ambient lighting and temperature of the recording environment.

48. The device of claim 45, wherein said distance sensor establishes a distance parameter of said sensing and image recording elements for a said recording session.

49. The device of claim 45, wherein said reference system includes said reference mark; said reference mark a laser projected onto a body portion adjacent said wound; an image of said projected reference mark stored for comparison with a projected reference mark of any said subsequent recording session.

50. The device of claim 49, wherein a projected image of said reference mark in a said subsequent recording session sensed by said imaging element is analysed by said system; said system indicating to a user when said projected image corresponds substantially with an image of said reference mark recorded in said first recording session.

51. The device of claim 45, wherein said sensing elements include temperature and ambient light sensors; said temperature and ambient light sensors establishing baseline parameters of said first recording session for comparison and adjustment of said parameters in any said subsequent recording session.

52. The device of claim 45, wherein said system compensates for ambient light conditions.

53. The device of claim 45, wherein said image recording elements include a digital camera.

54. The device of claim 53, wherein said digital camera is provided with a thermal imaging capability; said thermal imaging recording temperatures of said wound corrected according to variations from said base line parameter of ambient temperature.

55. The device of claim 45, wherein said system includes a view finder/display screen; said view finder/display screen acting in a first instance to display a subject wound sensed through a lens system of said digital camera; said display acting in a second instance to display simultaneously as a semi transparent overlay a previously recorded image of said subject wound and said subject wound sensed through said lens system.

56. The device of claim 46, wherein recorded sensed and image data is analysed by said system; analysis of said recorded data providing an output of progress of a said subject wound displayed on said view finder/display screen.

57. The device of claim 55, wherein said view finder/display screen is further adapted to the display of recorded textual data relating to treatment of a said wound.

58. The device of claim 55, wherein said sensing and said imaging elements and said view finder/display screen are incorporated in a single monitoring device.

59. The device of claim 55, wherein said sensing elements, said imaging elements and said view finder/display screen are separate devices; said separate devices connected to a central data processing unit.

60. A method of monitoring a wound; said method including the steps of:
(a) projecting a reference mark onto a surface area adjacent said wound,
(b) determining a distance between a sensing and recording device and said reference mark,
(c) establishing base line parameters of conditions under which parameters of said wound are recorded in a first sensing and image recording session,
(d) recording sensing and image data of said wound in subsequent sensing and image recording sessions,
(e) analysing differences between sensed and image data of a said subsequent sensing and image recording session with sensing and image data recorded in said first sensing and image recording session to derive an output of progress of said wound.

61. The method of claim 60, wherein said analysis is based on recorded temperature, colour and thermal imaging differences between said first recording session and said subsequent recording sessions.

62. The method of claim 60, wherein analysis and comparison of said sensing and image recordings of said first and subsequent recording sessions is provided by repeatability of parameters under which said sensing and image recording is conducted.

63. The method of claim 60, wherein repeatability of orientation and disposition parameters of sensing elements and imaging elements is provided by comparison of an image of a said projected reference mark with an image of said reference mark recorded in said first recording session.

64. The method of claims 60, wherein repeatability of sensing and imaging conditions of ambient light and temperature is provided by comparison of ambient light and temperature in a said subsequent recording session with corresponding ambient light and temperature recorded in said first recording session; said ambient light and temperature recorded in a said subsequent recording session compensated to correspond to said ambient light and temperature of said first recording session.
